(19) United States
(12) Patent Application Publication
(10) Pub. No.: US 2011/0077587 A1
(43) Pub. Date: Mar. 31, 2011

(54) METHOD AND APPARATUS FOR MANAGING JOINT IRRIGATION DURING HIP ARTHROSCOPY

(76) Inventors: James Flom, San Carlos, CA (US); Thomas Weisel, Ventura, CA (US)

(21) Appl. No.: 12/874,111

(22) Filed: Sep. 1, 2010

Related U.S. Application Data
(60) Provisional application No. 61/275,607, filed on Sep. 1, 2009.

(51) Int. Cl. A61M 1/00

(52) U.S. Cl. 604/30

(57) ABSTRACT

A fluid management system comprising:
an access cannula comprising:
   a shaft having a distal end and a proximal end, and a lumen extending between the distal end and the proximal end; and
   a septum disposed across the lumen; and
a pressure-sensitive valve in fluid communication with the lumen of the access cannula, the valve being connected to the lumen distal to the septum.

### REFERENCE TO PENDING PRIOR PATENT APPLICATION

This patent application claims benefit of pending prior U.S. Provisional Patent Application Ser. No. 61/275,607, filed Sep. 1, 2009 by James Flom et al. for METHOD AND APPARATUS FOR ACCESSING THE INTERIOR OF A HIP JOINT, INCLUDING THE PROVISION AND USE OF A NOVEL FLUID MANAGEMENT SYSTEM (Attorney's Docket No. FIAN-47 PROV), which patent application is hereby incorporated herein by reference.

### FIELD OF THE INVENTION

This invention relates to surgical methods and apparatus in general, and more particularly to surgical methods and apparatus for treating a hip joint.

### BACKGROUND OF THE INVENTION

### The Hip Joint in General

The hip joint is a ball-and-socket joint which movably connects the leg to the torso. The hip joint is capable of a wide range of different motions, e.g., flexion and extension, abduction and adduction, medial and lateral rotation, etc. See FIGS. 1A, 1B, 1C and 1D.

With the possible exception of the shoulder joint, the hip joint is perhaps the most mobile joint in the body. Significantly, and unlike the shoulder joint, the hip joint carries substantial weight loads during most of the day, in both static (e.g., standing and sitting) and dynamic (e.g., walking and running) conditions.

The hip joint is susceptible to a number of different pathologies. These pathologies can have both congenital and injury-related origins. In some cases, the pathology can be substantial at the outset. In other cases, the pathology may be minor at the outset but, if left untreated, may worsen over time. More particularly, in many cases, an existing pathology may be exacerbated by the dynamic nature of the hip joint and the substantial weight loads imposed on the hip joint.

The pathology may, either initially or thereafter, significantly interfere with patient comfort and lifestyle. In some cases, the pathology can be so severe as to require partial or total hip replacement. A number of procedures have been developed for treating hip pathologies short of partial or total hip replacement, but these procedures are generally limited in scope due to the significant difficulties associated with treating the hip joint.

A better understanding of various hip joint pathologies, and also the current limitations associated with their treatment, can be gained from a more thorough understanding of the anatomy of the hip joint.

### Anatomy of the Hip Joint

The hip joint is formed at the junction of the leg and the torso. More particularly, and looking now at FIG. 2, the head of the femur is received in the acetabular cup of the hip, with a plurality of ligaments and other soft tissue serving to hold the bones in articulating condition.

More particularly, and looking now at FIG. 3, the femur is generally characterized by an elongated body terminating, at its top end, in an angled neck which supports a hemispherical head (also sometimes referred to as “the ball”). As seen in FIGS. 3 and 4, a large projection known as the greater trochanter protrudes laterally and posteriorly from the elongated body adjacent to the neck of the femur. A second, somewhat smaller projection known as the lesser trochanter protrudes medially and posteriorly from the elongated body adjacent to the neck. An intertrochanteric crest (FIGS. 3 and 4) extends along the periphery of the femur, between the greater trochanter and the lesser trochanter.

Looking next at FIG. 5, the hip socket is made up of three constituent bones: the ilium, the ischium and the pubis. These three bones cooperate with one another (they typically ossify into a single “hip bone” structure by the age of 25 or so) in order to collectively form the acetabular cup. The acetabular cup receives the head of the femur.

Both the head of the femur and the acetabular cup are covered with a layer of articular cartilage which protects the underlying bone and facilitates motion. See FIG. 6.

Various ligaments and soft tissue serve to hold the ball of the femur in place within the acetabular cup. More particularly, and looking now at FIGS. 7 and 8, the ligamentum teres extends between the ball of the femur and the base of the acetabular cup. As seen in FIGS. 8 and 9, a labrum is disposed about the perimeter of the acetabular cup. The labrum serves to increase the depth of the acetabular cup and effectively establishes a suction seal between the ball of the femur and the rim of the acetabular cup, thereby helping to hold the head of the femur in the acetabular cup. In addition to the foregoing, and looking now at FIG. 10, a fibrous capsule extends between the neck of the femur and the rim of the acetabular cup, effectively sealing off the ball-and-socket members of the hip joint from the remainder of the body. The foregoing structures (i.e., the ligamentum teres, the labrum and the fibrous capsule) are encompassed and reinforced by a set of three main ligaments (i.e., the iliofemoral ligament, the ischiofemoral ligament and the pubofemoral ligament) which extend between the femur and the perimeter of the hip socket. See, for example, FIGS. 11 and 12, which show the iliofemoral ligament, with FIG. 11 being an anterior view and FIG. 12 being a posterior view.

### Pathologies of the Hip Joint

As noted above, the hip joint is susceptible to a number of different pathologies. These pathologies can have both congenital and injury-related origins.

By way of example but not limitation, one important type of congenital pathology of the hip joint involves impingement between the neck of the femur and the rim of the acetabular cup. In some cases, and looking now at FIG. 13, this impingement can occur due to irregularities in the geometry of the femur. This type of impingement is sometimes referred to as cam-type femoroacetabular impingement (i.e., cam-type FAI). In other cases, and looking now at FIG. 14, the impingement can occur due to irregularities in the geometry of the acetabular cup. This latter type of impingement is sometimes referred to as pincer-type femoroacetabular impingement (i.e., pincer-type FAI). Impingement can result in a reduced range of motion, substantial pain and, in some cases, significant deterioration of the hip joint.

By way of further example but not limitation, another important type of congenital pathology of the hip joint involves defects in the articular surface of the ball and/or the articular surface of the acetabular cup. Defects of this type sometimes start out fairly small but often increase in size over time, generally due to the dynamic nature of the hip joint and also due to the weight-bearing nature of the hip joint. Articular defects can result in substantial pain, induce and/or exacerbate arthritic conditions and, in some cases, cause significant deterioration of the hip joint.

By way of further example but not limitation, one important type of injury-related pathology of the hip joint involves trauma to the labrum. More particularly, in many cases, an accident or sports-related injury can result in the labrum being torn away from the rim of the acetabular cup, typically with a tear running through the body of the labrum. See FIG. 15. These types of injuries can be very painful for the patient and, if left untreated, can lead to substantial deterioration of the hip joint.

### The General Trend Toward Treating Joint Pathologies Using Minimally-Invasive, and Earlier, Interventions

The current trend in orthopedic surgery is to treat joint pathologies using minimally-invasive techniques. Such minimally-invasive, “keyhole” surgeries generally offer numerous advantages over traditional, “open” surgeries, including reduced trauma to tissue, less pain for the patient, faster recuperation times, etc.

By way of example but not limitation, it is common to re-attach ligaments in the shoulder joint using minimally-invasive, “keyhole” techniques which do not require large incisions into the interior of the shoulder joint. By way of further example but not limitation, it is common to repair torn meniscal cartilage in the knee joint, and/or to replace ruptured ACL ligaments in the knee joint, using minimally-invasive techniques.

While such minimally-invasive approaches can require additional training on the part of the surgeon, such procedures generally offer substantial advantages for the patient and have now become the standard of care for many shoulder joint and knee joint pathologies.

In addition to the foregoing, in view of the inherent advantages and widespread availability of minimally-invasive approaches for treating pathologies of the shoulder joint and knee joint, the current trend is to provide such treatment much earlier in the lifecycle of the pathology, so as to address patient pain as soon as possible and so as to minimize any exacerbation of the pathology itself. This is in marked contrast to traditional surgical practices, which have generally dictated postponing surgical procedures as long as possible so as to spare the patient from the substantial trauma generally associated with invasive surgery.

### Treatment for Pathologies of the Hip Joint

Unfortunately, minimally-invasive treatments for pathologies of the hip joint have lagged far behind minimally-invasive treatments for pathologies of the shoulder joint and the knee joint. This is generally due to (i) the constrained geometry of the hip joint itself, and (ii) the nature and location of the pathologies which must typically be addressed in the hip joint.

More particularly, the hip joint is generally considered to be a “tight” joint, in the sense that there is relatively little room to maneuver within the confines of the joint itself. This is in marked contrast to the shoulder joint and the knee joint, which are generally considered to be relatively “spacious” joints (at least when compared to the hip joint). As a result, it is relatively difficult for surgeons to perform minimally-invasive procedures on the hip joint.

Furthermore, the pathways for entering the interior of the hip joint (i.e., the natural pathways which exist between adjacent bones and/or delicate neurovascular structures) are generally much more constraining for the hip joint than for the shoulder joint or the knee joint. This limited access further complicates effectively performing minimally-invasive procedures on the hip joint.

In addition to the foregoing, the nature and location of the pathologies of the hip joint also complicate performing minimally-invasive procedures on the hip joint. By way of example but not limitation, consider a typical detachment of the labrum in the hip joint. In this situation, instruments must generally be introduced into the joint space using an angle of approach which is offset from the angle at which the instrument addresses the tissue. This makes drilling into bone, for example, significantly more complicated than where the angle of approach is effectively aligned with the angle at which the instrument addresses the tissue, such as is frequently the case in the shoulder joint. Furthermore, the working space within the hip joint is typically extremely limited, further complicating repairs where the angle of approach is not aligned with the angle at which the instrument addresses the tissue.

As a result of the foregoing, minimally-invasive hip joint procedures are still relatively difficult to perform and relatively uncommon in practice. Consequently, patients are typically forced to manage their hip pain for as long as possible, until a resurfacing procedure or a partial or total hip replacement procedure can no longer be avoided. These procedures are generally then performed as a highly-invasive, open procedure, with all of the disadvantages associated with highly-invasive, open procedures.

As a result, there is, in general, a pressing need for improved methods and apparatus for treating pathologies of the hip joint.

### Arthroscopic Access to the Interior of the Hip Joint

Successful hip arthroscopy generally requires safe and effective access to the interior of the hip joint. More particularly, successful hip arthroscopy generally requires the creation of a plurality of access portals which extend inwardly from the surface of the skin, down to the interior of the hip joint, extending through the intervening layers of tissue, including skin, fat, muscle and capsule tissue. These access portals may also continue down to the specific surgical site within the interior of the hip joint. Depending on the specific surgical site which is to be accessed within the interior of the hip joint, different anatomical pathways may be utilized for the access portals. By way of example but not limitation, one anatomical pathway may be used where a torn labrum is to be repaired, and another anatomical pathway may be used where the lesser trochanter must be addressed. And, in most cases, multiple access portals are required, with one access portal being used for visualization (i.e., to introduce an arthroscope into the interior of the hip joint), while other access portals are used for irrigation and to pass surgical instruments to and from the surgical site, etc.

Establishing these access portals typically involves forming an opening from the top surface of the skin down to the interior of the joint, and lining that opening with a tubular liner (sometimes referred to as an “access cannula”). This access cannula holds the incision open and provides a surgical pathway (or “corridor”) from the top surface of the skin down to the interior of the hip joint, thereby enabling instrumentation (e.g., arthroscopes, surgical instruments, etc.) to be passed through the central lumen of the access cannula so as to reach the remote surgical site within the joint. Thus the provision and use of access cannulas are generally an important aspect of enabling minimally-invasive, “keyhole” surgery to be performed on the hip joint.

See, for example, FIG. 16, which shows multiple access cannulas extending into a hip joint during an arthroscopic hip procedure.

### Joint Irrigation

During arthroscopic surgery, it is common to irrigate the joint with a flow of fluid (e.g., saline) so as to rinse away blood and other debris from the surgical site and maintain a clear surgical field. To this end, one access cannula is typically used to introduce irrigation fluid into the joint, and another access cannula is typically used to vent irrigation fluid (and debris) from the joint.

In addition, it is also common to introduce the irrigation fluid into the joint under pressure, so as to “inflate” the joint and thereby improve access to, and visualization of, the patient's tissue. To this end, the irrigation fluid is typically delivered to the joint under the pressure of a gravity feed or a mechanical pump.

Unfortunately, the joint is not a true watertight structure, so it is possible for the irrigation fluid to migrate through the lining of the joint and into adjacent tissue and/or anatomical spaces. This is particularly true where the irrigation fluid is being introduced into the joint under pressure so as to “inflate” the joint. In most cases, this extravasated fluid is relatively modest in quantity and is easily absorbed away by the body, with no negative consequences for the patient. However, where larger amounts of irrigation fluid extravasate out of the joint and into adjacent tissue and/or anatomical spaces, patient discomfort can be substantial. Indeed, in extreme cases, the quantity of extravasated fluid can be so large as to impose a substantial health risk to the patient. By way of example but not limitation, where large amounts of fluid extravasate out of the hip joint and into the abdomen, the accumulated fluid can interfere with normal bodily function (“abdominal compartment syndrome”) and pose serious health risks for the patient.

On account of the foregoing, the surgeon generally tries to minimize fluid extravasation by (i) keeping the irrigation fluid at a modest but sufficient pressure, and (ii) minimizing the time duration of the surgery.

Unfortunately, it can be difficult for the surgeon to keep the pressure of the irrigation fluid at a modest but sufficient pressure, even where a constant flow mechanical pump is being used to introduce the irrigation fluid into the joint. This is because many surgical instruments use suction to clear debris from the surgical field, and this suction dynamically changes the pressure of the irrigation fluid within the joint.

By way of example but not limitation, a powered rotary shaver is often used to excise tissue (including bone) from within a joint. The powered rotary shaver typically provides suction at its working tip in order to remove the excised tissue from the surgical site. Thus, when the powered rotary shaver is operating, a substantial amount of irrigation fluid is being pulled from the joint through the suction of the powered rotary shaver; however, when the powered rotary shaver is not operating, little or no irrigation fluid is being pulled from the joint through the powered rotary shaver. Accordingly, where a powered rotary shaver is being used within a joint, the fluid pressure within the joint tends to vary with the operation of the powered rotary shaver, i.e., the fluid pressure is low when the powered rotary shaver is operating (and pulling irrigation fluid out of the joint) and the fluid pressure is high when the powered rotary shaver is not operating (and hence not pulling irrigation fluid out of the joint).

Thus it will be seen that the operation of a powered rotary shaver (or other suctioning instrument) within the joint can make it difficult to maintain a desired fluid pressure within the joint.

Additionally, at times when the fluid flow out of the joint is increased by, for example, the use of a suctioning instrument, the mechanical pump will typically increase fluid flow into the joint in order to compensate for the increased fluid drain. However, if the fluid exit path out of the joint is thereafter suddenly closed (e.g., if the suctioning instrument is thereafter turned off), the fluid pressure in the joint can unintentionally spike, which can result in damage to the tissue within the joint and/or exacerbate the fluid extravasation problems discussed above.

Furthermore, if the fluid path out of the joint should become unexpectedly blocked (e.g., by a large piece of debris lodging in a suctioning instrument and/or in a fluid-venting cannula), then fluid flow out of the joint may be significantly reduced, which may also lead to a rapid increase in fluid pressure within the joint. Therefore, it is typically desirable to have multiple fluid exit paths out of the joint, so that adequate fluid flow out of the joint can be maintained even if one fluid exit path should become blocked, whereby to avoid fluid pressure spikes within the joint. At the same time, however, the provision of too many fluid exit paths out of a joint can result in excessive trauma to the patient's tissue and can make it difficult to establish an adequate “joint-inflating” pressure within the joint.

In addition to the foregoing, it should also be appreciated that the cannula which allows fluid to enter the joint will itself typically restrict fluid flow into the joint. More specifically, the level of fluid restriction provided by the cannula depends upon the size and length of the fluid path passing through the cannula. Thus cannulas of different sizes provide fluid flow restrictions of different magnitudes. However, surgeons typically select from a wide range of different cannulas, depending upon the specific procedure to be performed, the specific instruments to be used, etc. The mechanical pump used to introduce the irrigation fluid into the joint is typically not able to detect the level of fluid restriction provided by the specific cannula selected by the surgeon, and therefore cannot exactly compensate for the level of fluid restriction provided by that specific cannula. The surgeon, then, typically sets the pump at a rate estimated to be correct for the conditions at hand, but in practice the surgeon does not know the actual pressure within the joint. This can create situations where either: (1) not enough, or too much, irrigation fluid is flowing into the joint, which impacts visualization, or patient safety, respectively; and/or (2) the fluid pressure within the joint is higher or lower than desired, which can impact patient safety, and/or visualization, respectively.

Thus there is a need for a novel fluid management system which can manage the pressure of an irrigation fluid during an arthroscopic surgical procedure. This fluid management system should, preferably, provide good visualization while minimizing safety concerns. The fluid management system should, preferably, be capable of providing an appropriate flow of irrigation fluid at all times, with the irrigation fluid being delivered at a high enough pressure to provide good visualization but at a low enough pressure to minimize safety concerns. The fluid management system should, preferably, also allow fluid flow to be essentially stopped when necessary, but still minimize pressure variation within the joint. And the fluid management system should, preferably, also accommodate pressure variations due to the use of a variety of instruments, including those with and without suction and, where suction is used, where that suction is variable in time and/or intensity. The fluid management system should, preferably, also accommodate the pressure fluctuations associated with the use of a mechanical pump, if one is used.

### SUMMARY OF THE PRESENT INVENTION

These and other objects of the present invention are addressed by the provision and use of a novel fluid management system for managing the pressure of an irrigation fluid during an arthroscopic surgical procedure. The novel fluid management system permits the pressure of the fluid within a joint to be managed during an arthroscopic procedure so as to minimize fluid extravasation, among other things.

In one form of the present invention, there is provided a fluid management system comprising:

an access cannula comprising:
a shaft having a distal end and a proximal end, and a lumen extending between the distal end and the proximal end; and
a septum disposed across the lumen; and

a pressure-sensitive valve in fluid communication with the lumen of the access cannula, the valve being connected to the lumen distal to the septum.

In another form of the present invention, there is provided a method for managing fluid flow in an arthroscopic procedure, the method comprising:

providing a fluid management system comprising:
an access cannula comprising:
   a shaft having a distal end and a proximal end, and a lumen extending between the distal end and the proximal end; and
   a septum disposed across the lumen; and
a pressure-sensitive valve in fluid communication with the lumen of the access cannula, the valve being connected to the lumen distal to the septum;

deploying the access cannula within the body so that the distal end of the access cannula is disposed within an anatomical space; and

introducing fluid into the anatomical space so that the fluid enters the lumen of the access cannula and communicates with the pressure-sensitive valve.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIGS. 1A-1D are schematic views showing various aspects of hip motion;

FIG. 2 is a schematic view showing bone structures in the region of the hip joint;

FIG. 3 is a schematic anterior view of the femur;

FIG. 4 is a schematic posterior view of the top end of the femur;

FIG. 5 is a schematic view of the pelvis;

FIGS. 6-12 are schematic views showing bone and soft tissue structures in the region of the hip joint;

FIG. 13 is a schematic view showing cam-type femoroacetabular impingement (i.e., cam-type FAI);

FIG. 14 is a schematic view showing pincer-type femoroacetabular impingement (i.e., pincer-type FAI);

FIG. 15 is a schematic view showing a labral tear;

FIG. 16 is a schematic view showing multiple access cannulas being used in a hip arthroscopy procedure;

FIGS. 17 and 18 are schematic views showing a prior art access cannula;

FIG. 19 is a schematic view showing another prior art access cannula;

FIG. 20 is a schematic view showing a fluid management system formed in accordance with the present invention;

FIG. 21 is a schematic view showing another fluid management system formed in accordance with the present invention;

FIGS. 22-24 are schematic views showing the fluid management system of FIG. 21 incorporating a stopcock;

FIG. 25 is a schematic view showing another fluid management system formed in accordance with the present invention; and

FIG. 26 is a schematic showing another fluid management system formed in accordance with the present invention.

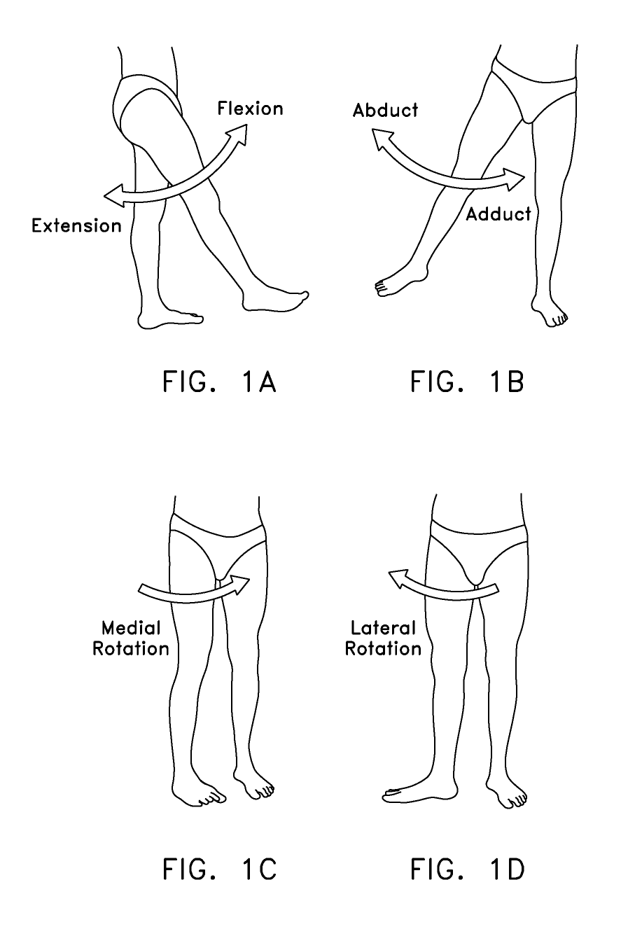

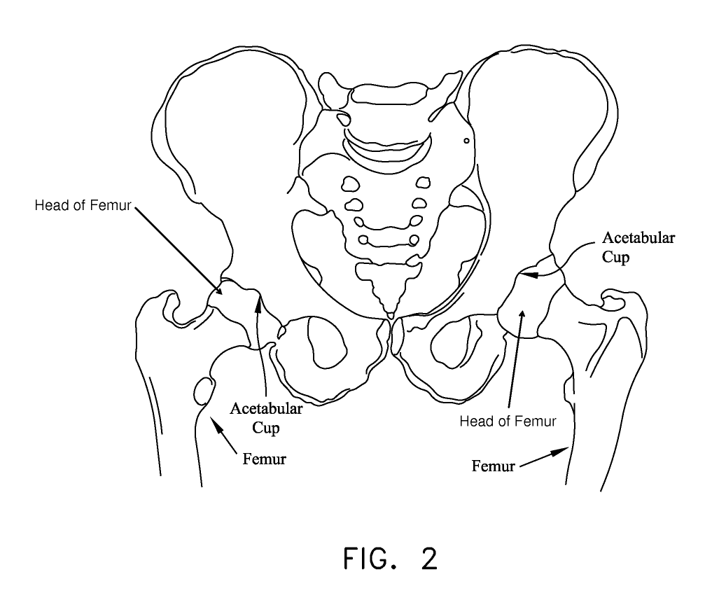

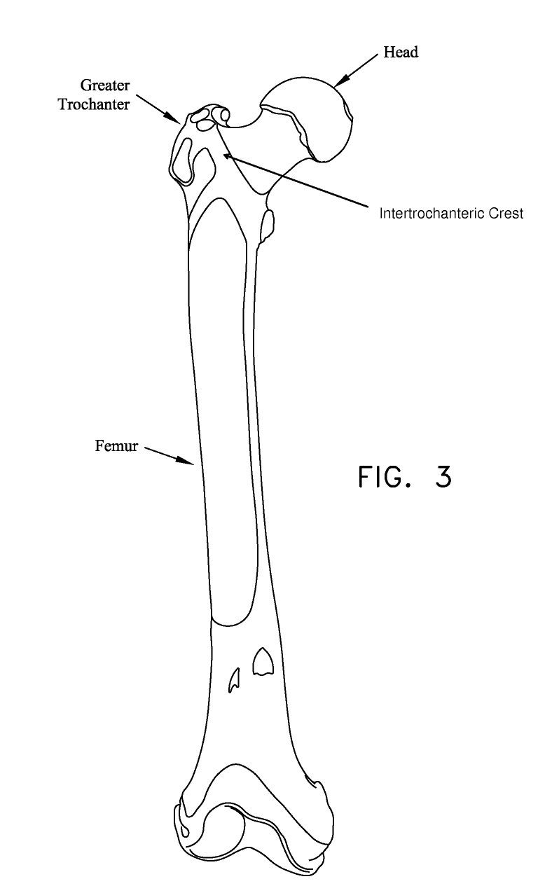

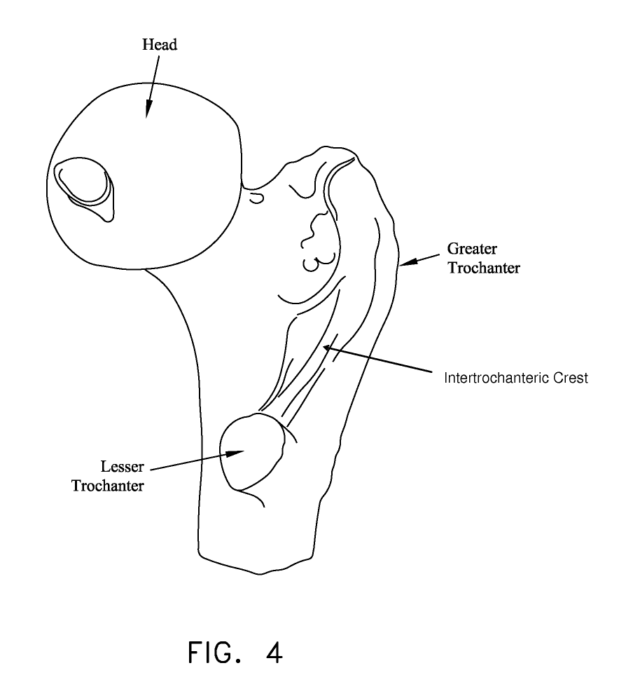

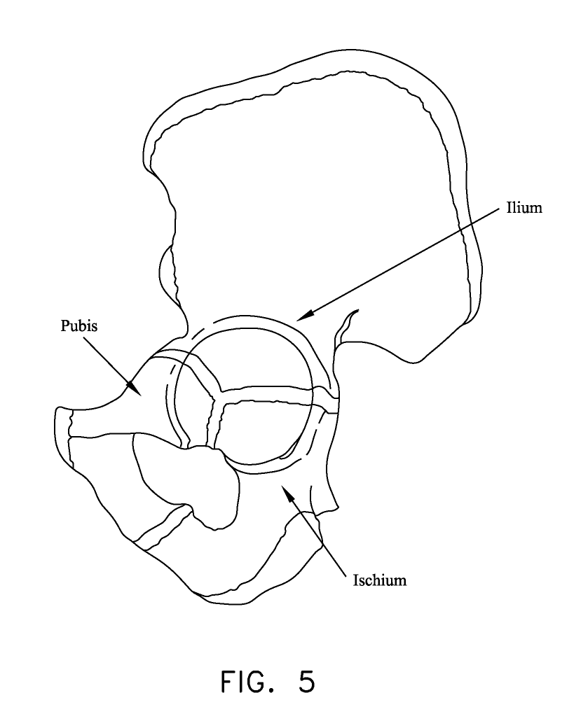

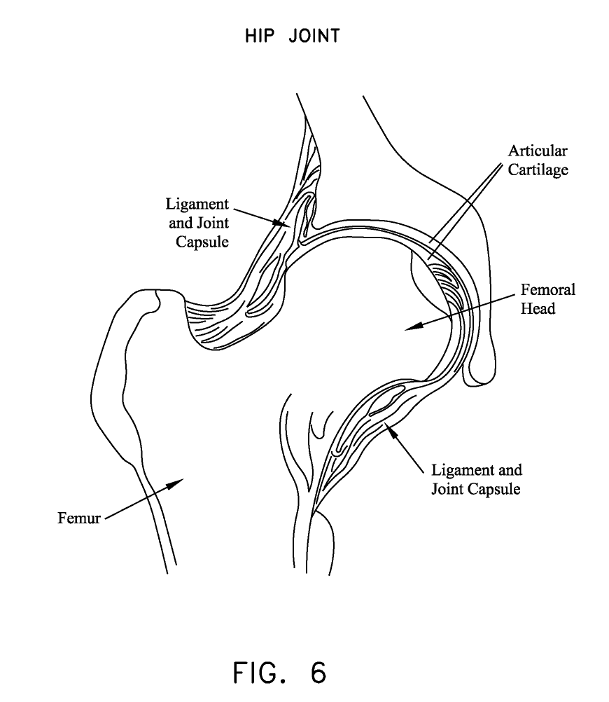

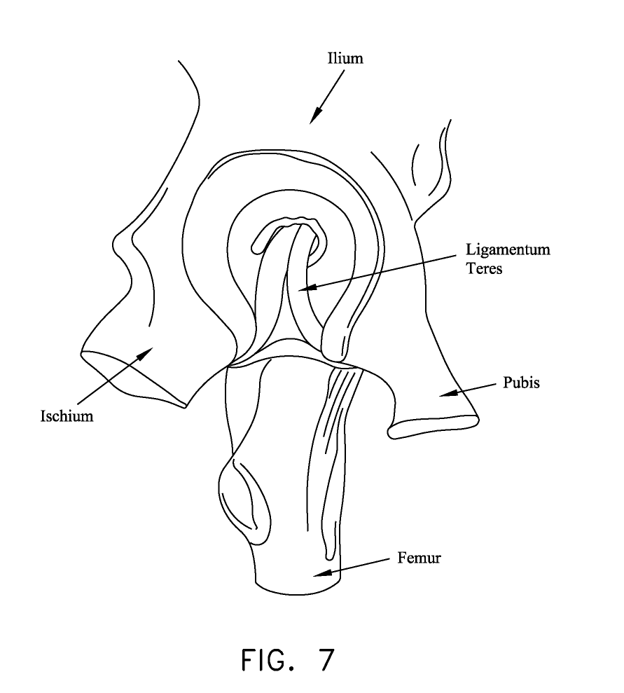

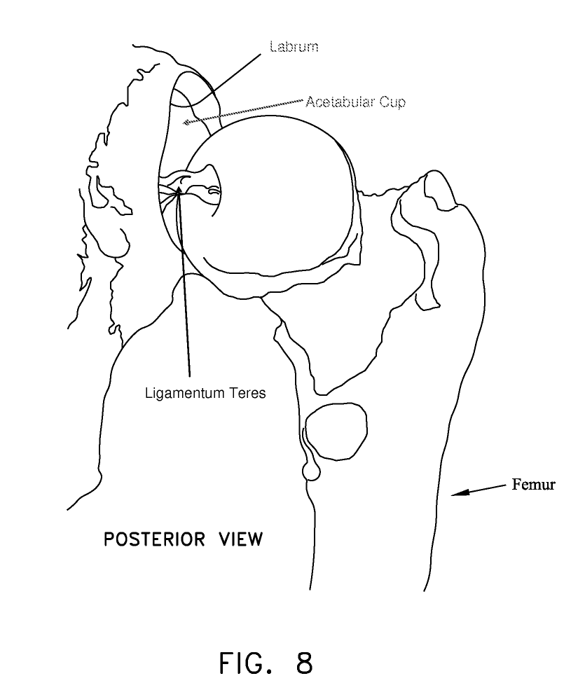

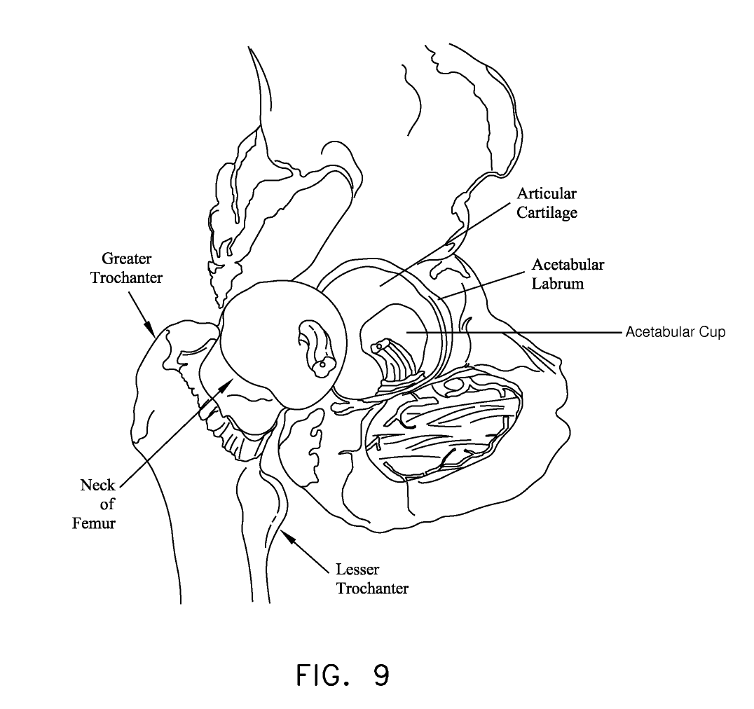

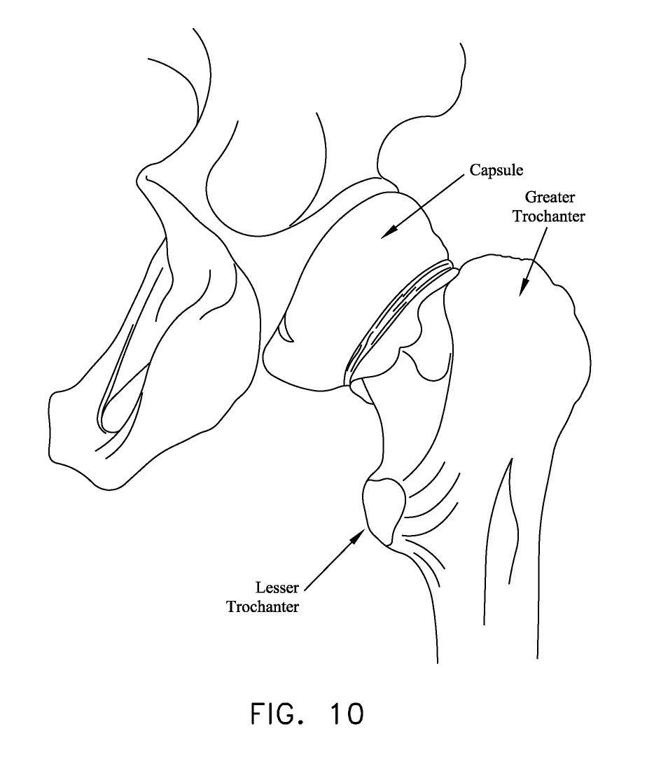

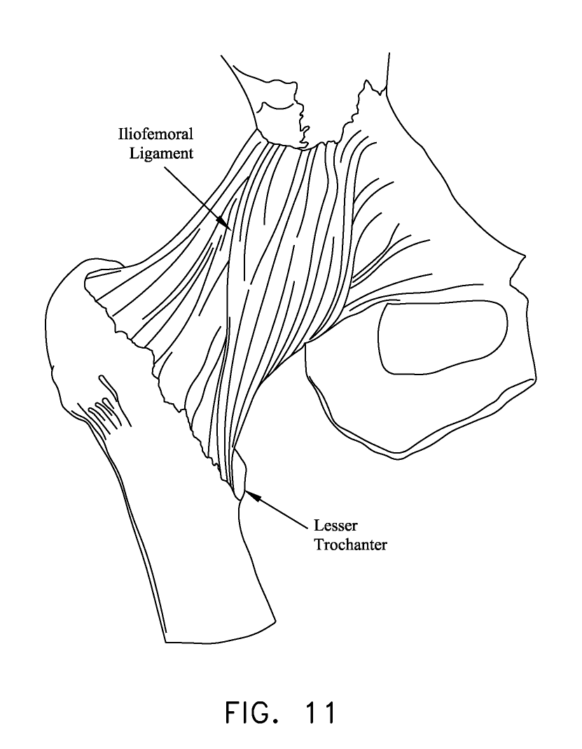

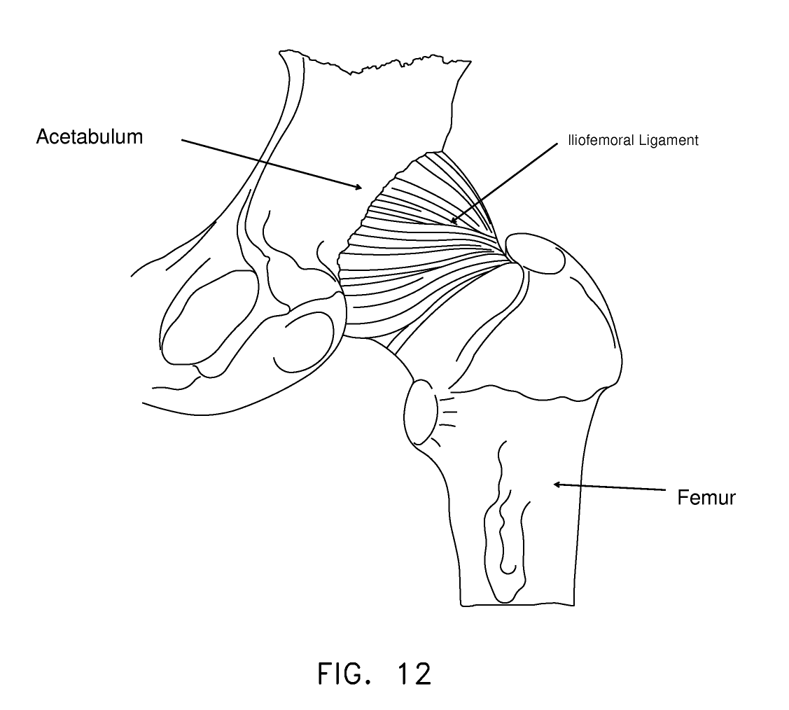

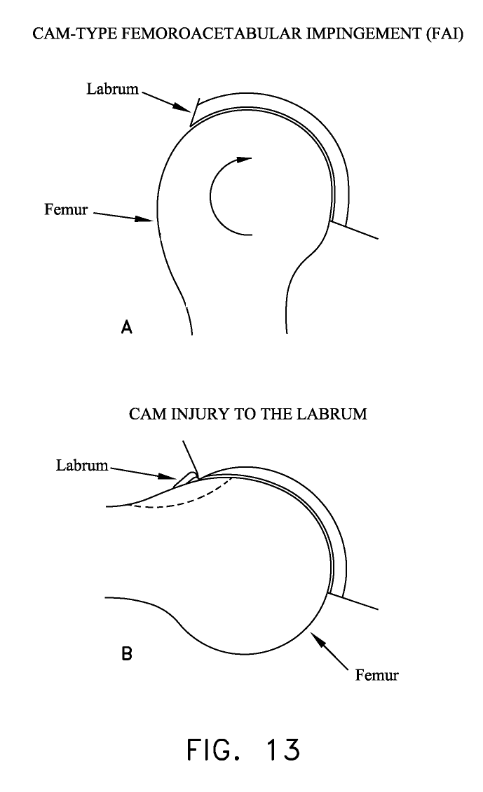

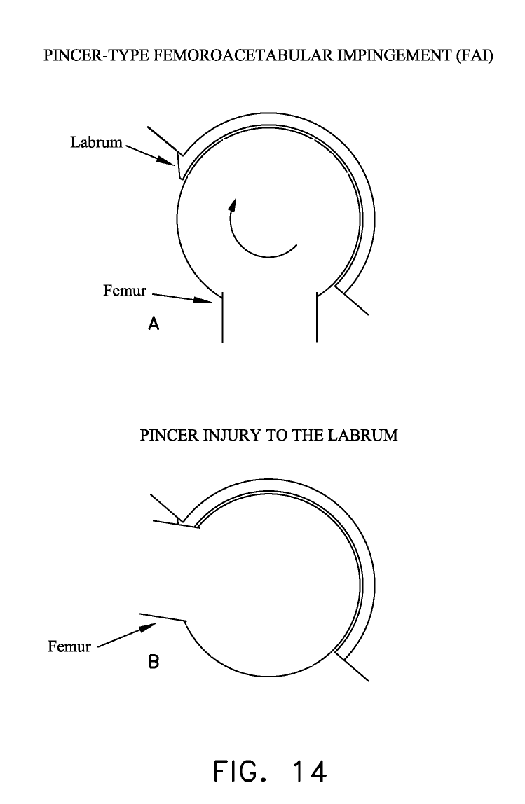

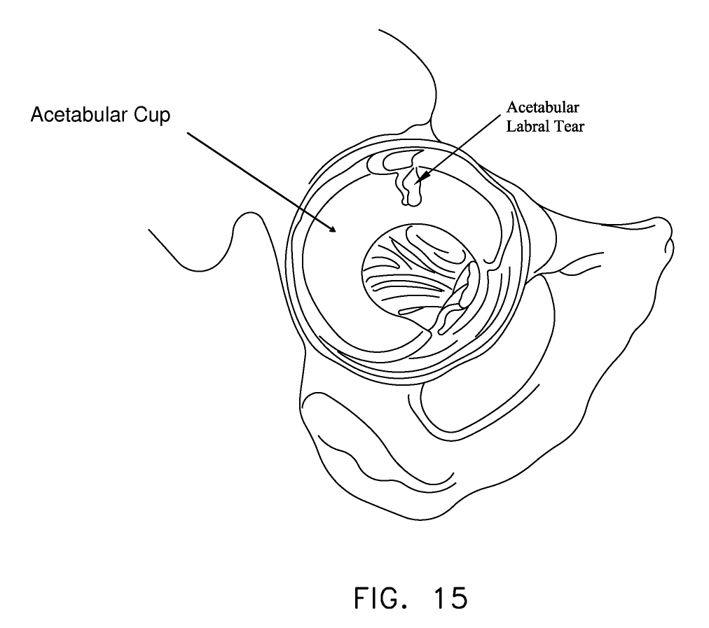

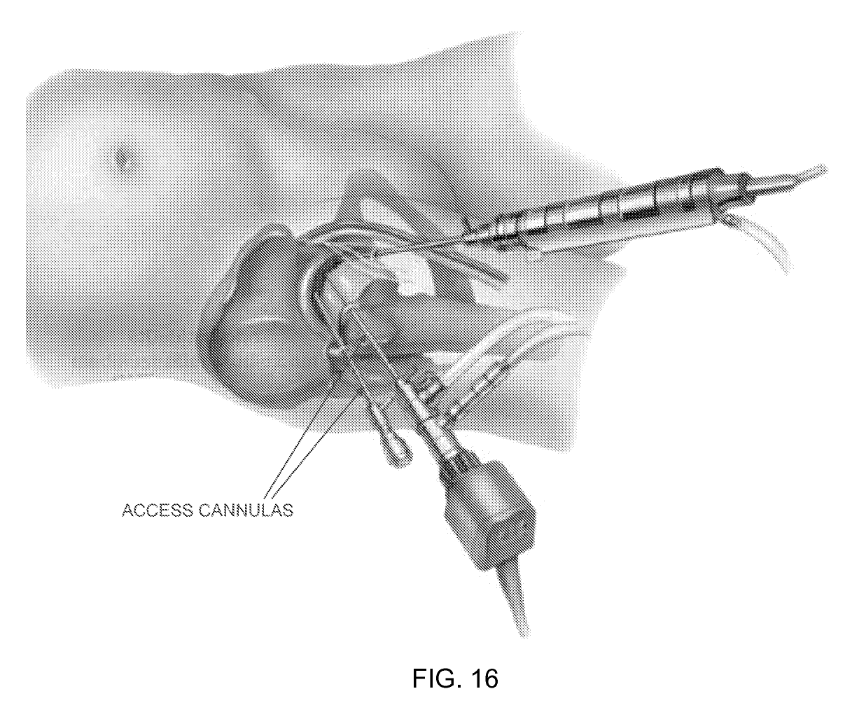

### DETAILED DESCRIPTION OF THE PREFERRED EMBODIMENTS

The present invention provides a novel fluid management system for managing the pressure of an irrigation fluid during an arthroscopic surgical procedure. The novel fluid management system permits the pressure of the fluid within a joint to be managed during an arthroscopic procedure so as to minimize fluid extravasation, among other things.

In accordance with the present invention, there is provided a novel surgical cannula which comprises a valve connected to an outlet on the surgical cannula such that certain fluid functions associated with the surgical cannula can be managed by operation of the valve.

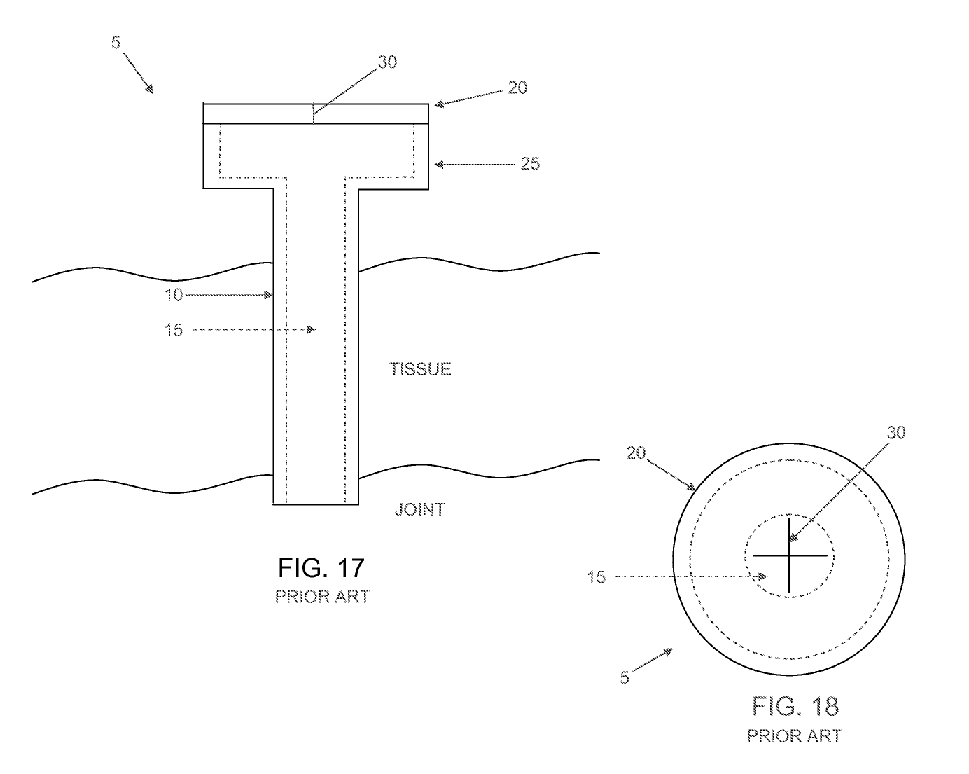

More particularly, and looking now at FIGS. 17 and 18, there is shown a typical prior art surgical cannula 5 of the sort currently used during arthroscopic surgery. For the purposes of the present discussion, the present invention will be discussed in the context of an arthroscopic cannula, but it should be appreciated that the invention is equally applicable to other types of surgical cannulas, e.g., a surgical cannula of the type used for laparoscopic surgery. These prior art surgical cannulas are generally characterized by a conduit or tube 10 having a central lumen 15 which communicates with the interior of the joint (or other anatomical space) which, as discussed above, is typically pressurized with irrigation fluid during surgery. A dam or septum 20 is typically provided at the proximal end of the cannula, e.g., at the cannula head 25. This dam or septum 20 substantially restricts the free flow of irrigation fluid out the joint, but is slit (e.g., at 30) so as to allow instruments to be passed through the dam or septum 20, down central lumen 15 and into the joint.

As noted above, it is often desired by the doctor to have a certain amount of fluid flow through the joint space in order to keep the viewing field clear of debris and blood. If the dam or septum 20 works perfectly, there is little or no pathway for fluid to escape out the top of the cannula.

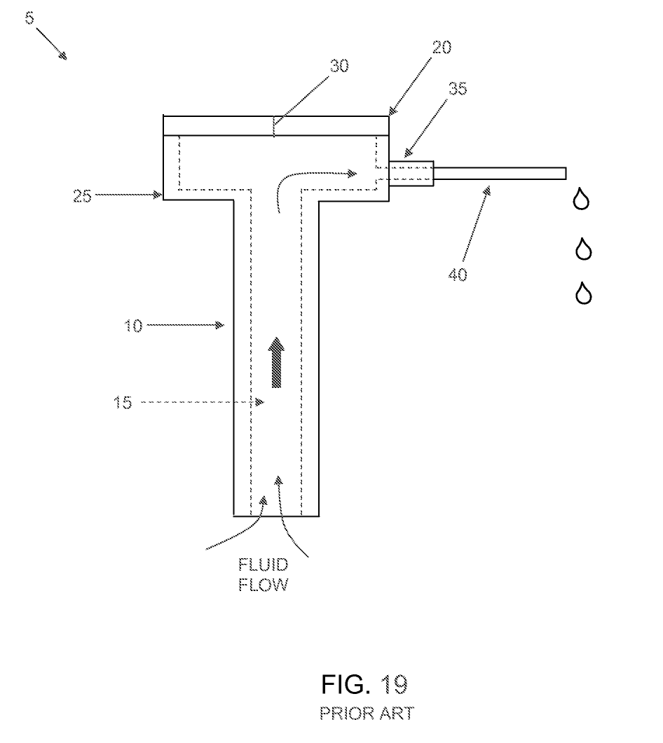

Accordingly, and looking now at FIG. 19, it is common in prior art cannulas to provide an opening (or exit port) 35 in the surgical cannula, distal to the dam (or septum) 20 and proximal to the skin, in order to allow the desired outflow of irrigation fluid to occur at all times. For purposes of illustration but not limitation, in FIG. 19 a tube 40 is shown added to the surgical cannula at the opening (or exit port) 35. On account of this construction, fluid can continuously leak out of the surgical cannula 5 via the opening (or exit port) 35, such that the cannula can vent irrigation fluid out of the joint space.

Unfortunately, the prior art construction shown in FIG. 19 suffers from the fact that it may “waste” a sizable quantity of fluid during times that outflow is not required. Furthermore, it allows fluid venting to occur at times when fluid venting may not be desired, e.g., such as when a suction instrument is working within the joint and it may be desirable to minimize cannula outflow so as to maintain joint “inflation”.

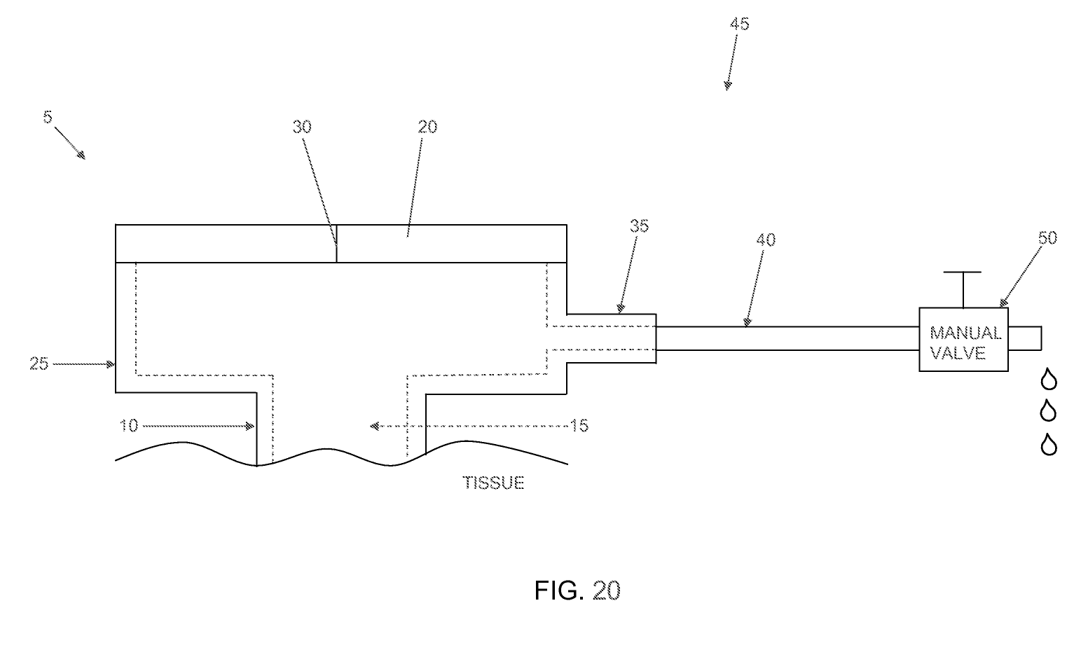

To that end, in accordance with the present invention, and looking now at FIG. 20, there is shown a novel fluid management system 45 which comprises the surgical cannula 5 having an opening (or port) 35 and tube 40, and also having a manual valve 50 located on the end of tube 40. Manual valve 50 is constructed so that it can be turned “on” and “off” as flow requirements dictate. Manual valve 50 can be a bi-state valve (i.e., only “on” or only “off”) or variable-state valve (i.e., so that different flow rates can be set by the surgeon as desired).

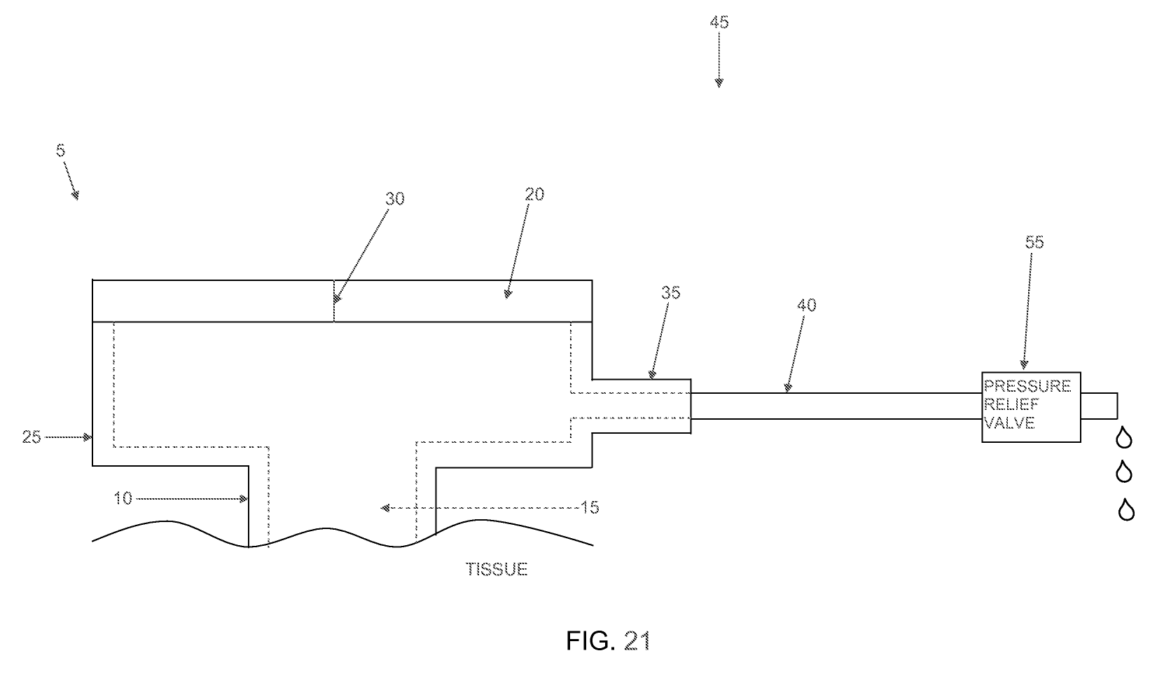

Looking next at FIG. 21, it is also possible to provide fluid management system 45 with a pressure relief valve 55 in place of the aforementioned manual valve 50. Pressure relief valve 55 is constructed so that it will automatically (i) open when the pressure differential on the two sides of the valve rises above a pre-determined value, and (ii) close when the pressure differential on the two sides of the valve falls below a pre-determined value (note that the pre-determined valve closing value does not necessarily need to be the same value as the pre-determined valve opening value). Thus, in this form of the invention, pressure relief valve 55 is intended to be essentially a bi-state device (i.e., “open” or “closed”). Such a construction provides the significant advantage that the valve can open to relieve pressure if the pressure within the joint rises too high (which can cause extravasation), but otherwise remain closed so as to minimize waste from excess fluid flow or to help maintain “inflation” pressure when a suction instrument is being used within the joint. In the latter situation, the one-way valve would also restrict the inflow of air into the joint in case the joint pressure should fall too low.

Significantly, by setting the operational parameters of pressure relief valve 55 with a consideration of desired operating flows, pressure relief valve 55 can function as more than just a safety mechanism to prevent excess fluid pressure from building up within the joint. More particularly, this construction can be advantageous when used in conjunction with certain arthroscopic instruments which are connected to suction; for example, shavers and burrs. During their use, the suction associated with these devices will lower the fluid pressure in the joint. For example, the suction from a powered rotary shaver can lower the joint pressure from about 60 mm Hg to about 20 mm Hg. This can cause undesirable effects such as, but not limited to, the capsule collapsing and impairing the field of view. Ideally, the joint pressure remains constant while the suctioning instrument is being used. A pressure relief valve which is normally open, but which closes at the desired joint pressure threshold, could be used to achieve this. This can be achieved by selecting the operational parameters of pressure relief valve 55 so that it is normally open (and venting fluid) when the joint is in its normal “inflated” condition, but which closes when a suction instrument causes joint pressure to fall below a pre-determined minimum.

By way of example but not limitation, if the mechanical irrigation pump is set at 50 mm Hg of pressure, the surgeon may want the fluid within the joint to remain at 40 mm Hg of pressure. This means that, theoretically, there is flow coming out of pressure relief valve 55 when nothing else is occurring, keeping the joint pressure low and the field of view clear. If a powered rotary shaver is then activated within the joint, it may require large amounts of fluid to be added to the joint in order to maintain the desired pressure, and may result in the joint pressure falling too low if the pump cannot supply enough compensating fluid flow. For this situation, pressure relief valve 55 can close and substantially all fluid flow is available to accommodate the suction of the powered rotary shaver without loss of joint “inflation”.

Examples of pressure relief valves that have a set pressure to regulate flow are: (i) spring and ball valves, and (ii) duck bill valves.

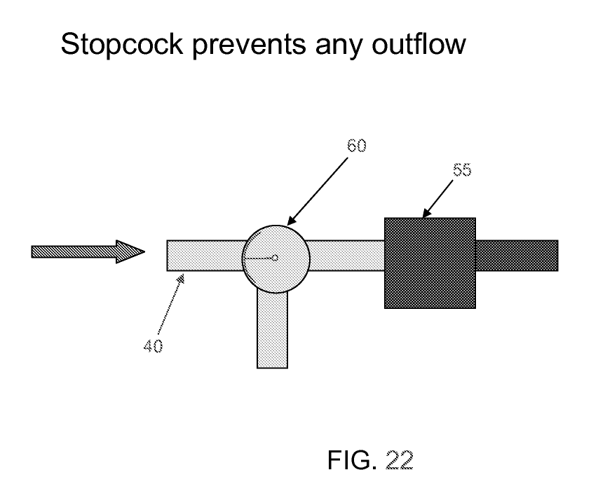

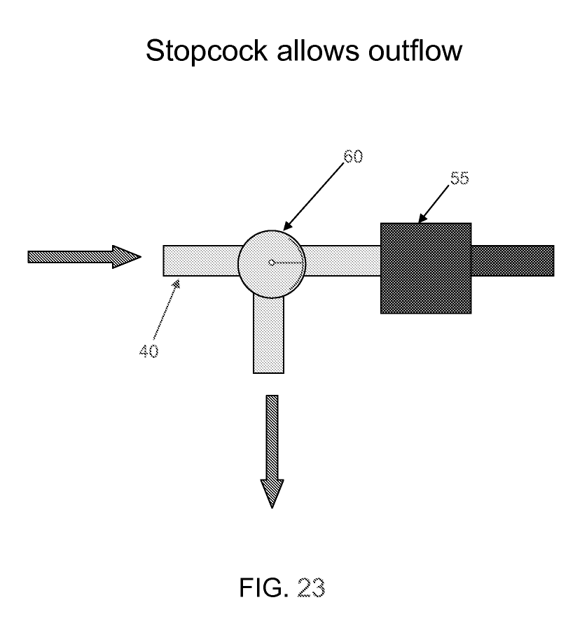

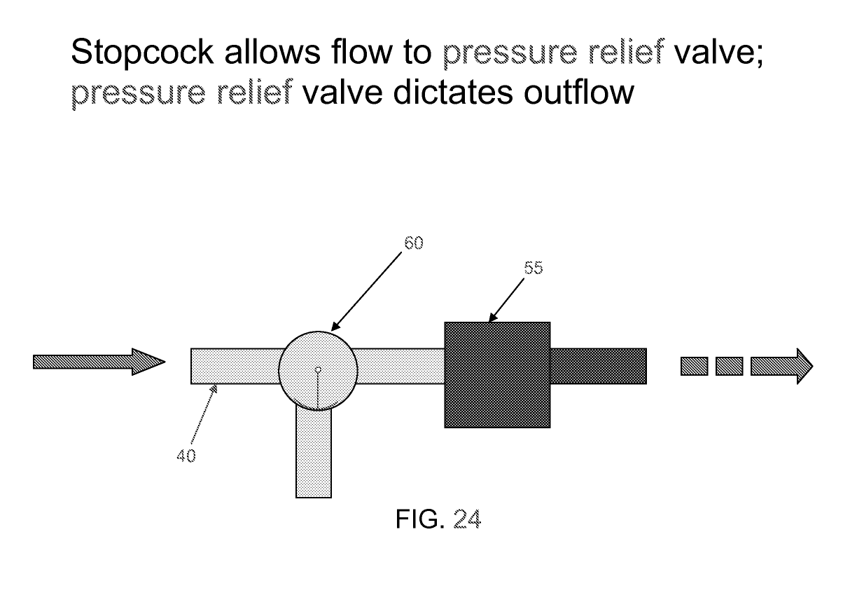

Pressure relief valve 55 can be incorporated directly into the outflow line 40, e.g., such as is shown in FIG. 21, or it can be incorporated directly into outflow port 35, or it can be incorporated into another portion of the surgical cannula, e.g., at or in place of the dam or septum 20. Alternatively, pressure relief valve 55 can be combined with a stopcock or other valve so as to provide the physician with the option of choosing the preferred flow option. For example, if pressure relief valve 55 were attached to one outlet on a 3-way stopcock, the physician has the choice of no-flow, (variable) flow and pressure relief flow. See, for example, FIGS. 22-24: FIG. 22 shows a stopcock 60 set at the no-flow position; FIG. 23 shows the stopcock set at the (variable) flow position; and FIG. 24 shows the stopcock set in the pressure relief flow condition.

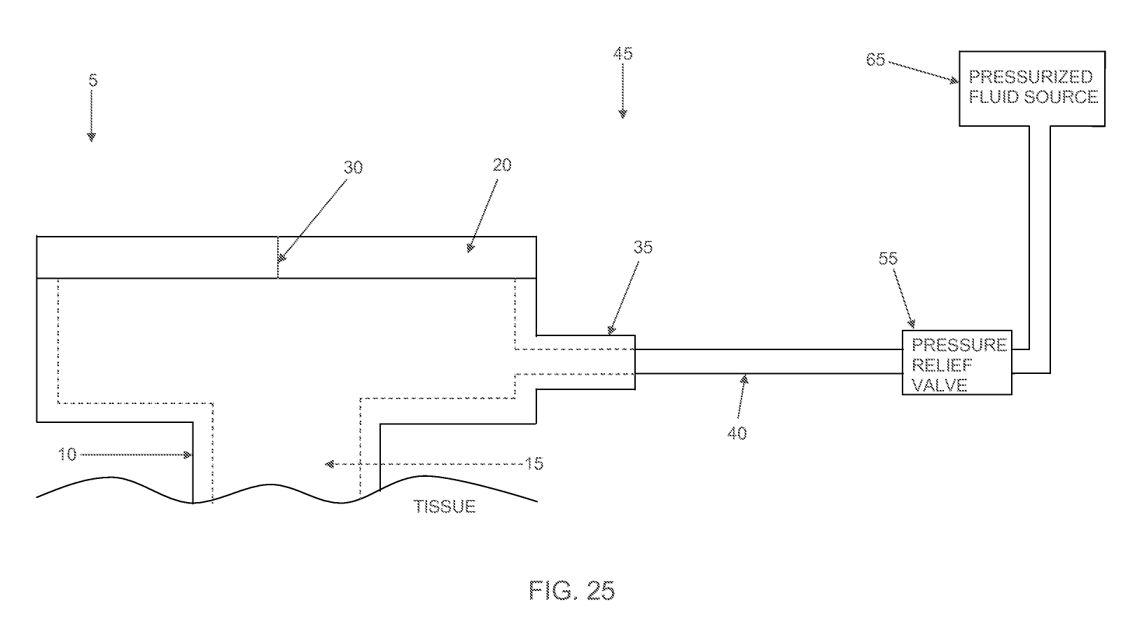

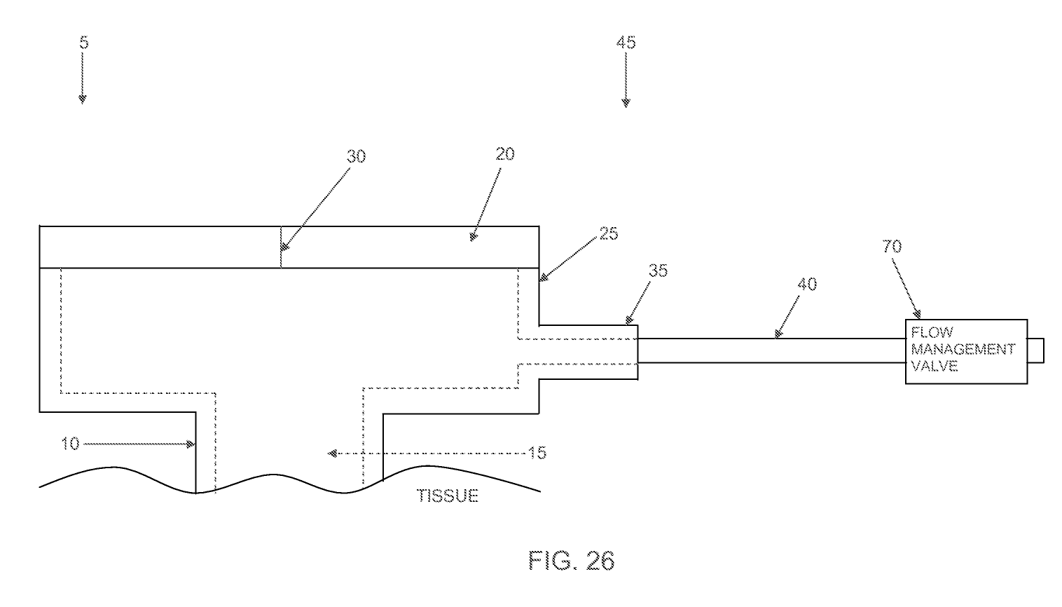

If desired, and looking now at FIG. 25, pressure relief valve 55 can be interposed between line 35 and a pressurized fluid source 65, with pressure relief valve 55 set so that if the pressure within the joint falls below a certain threshold pressure, the pressure relief valve opens to permit fluid to flow into the joint, whereby to maintain fluid pressure within the joint.

In an alternative form of the invention, and looking now at FIG. 26, pressure relief valve 55 can be replaced by a flow management valve 70. Flow management valve 70 is arranged so that the amount that the valve is open or closed depends on the pressure differential across the valve (e.g., a greater pressure differential creates a more open valve and hence more fluid flow, a smaller pressure differential creates a less open valve and hence less fluid flow). With a valve of this sort, by properly selecting the operational parameters of the valve, the valve can normally be venting a limited amount of fluid (e.g., 50% of the total flow capacity of the valve) from the joint; if a suction instrument should thereafter be activated so as to cause fluid pressure within the joint to fall, the valve can automatically close down to restrict fluid flow (e.g., to 0% of the total flow capacity of the valve) and help maintain fluid pressure within the joint; and if the fluid pressure within the joint should suddenly spike, the valve can automatically open more (e.g., to 100% of the total flow capacity of the valve) to help reduce the pressure within the joint. In a preferred form of the invention, flow management valve 70 is continuously variable between its totally open state and its totally closed state.

### Use of the Novel Fluid Management System for Other Applications

It should be appreciated that the novel fluid management system of the present invention may be used for regulating fluid within other joints in the body (e.g., the shoulder, the knee, etc.), and/or for regulating fluid within other locations in the body (e.g., within the abdominal cavity).

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A fluid management system comprising:
an access cannula comprising:
a shaft having a distal end and a proximal end, and a lumen extending between the distal end and the proximal end; and
a septum disposed across the lumen; and
a pressure-sensitive valve in fluid communication with the lumen of the access cannula, the valve being connected to the lumen distal to the septum.

2. A fluid management system according to claim 1 wherein the pressure-sensitive valve comprises a pressure relief valve.

3. A fluid management system according to claim 2 wherein the pressure relief valve is a bi-state valve.

4. A fluid management system according to claim 1 wherein the pressure-sensitive valve comprises a flow management valve.

5. A fluid management system according to claim 4 wherein the flow management valve is a continuously variable valve.

6. A method for managing fluid flow in an arthroscopic procedure, the method comprising:
providing a fluid management system comprising:
an access cannula comprising:
a shaft having a distal end and a proximal end, and a lumen extending between the distal end and the proximal end; and
a septum disposed across the lumen; and
a pressure-sensitive valve in fluid communication with the lumen of the access cannula, the valve being connected to the lumen distal to the septum;
deploying the access cannula within the body so that the distal end of the access cannula is disposed within an anatomical space; and
introducing fluid into the anatomical space so that the fluid enters the lumen of the access cannula and communicates with the pressure-sensitive valve.
